(19) United States
(12) Patent Application Publication
(10) Pub. No.: US 2021/0085306 A1
(43) Pub. Date: Mar. 25, 2021

(54) RETRACTOR

(71) Applicant: Spinal Elements, Inc., Carlsbad, CA (US)

(72) Inventors: Kristen Clauss, Encinitas, CA (US); David Ortiz, Oceanside, CA (US); Jason Blain, Encinitas, CA (US)

(21) Appl. No.: 17/022,547

(22) Filed: Sep. 16, 2020

Related U.S. Application Data
(60) Provisional application No. 62/903,318, filed on Sep. 20, 2019.

(51) Int. Cl. A61B 17/02; A61M 29/00

(52) U.S. Cl. CPC A61B 17/0206; A61B 17/025; A61B 2017/00915; A61B 2017/0256; A61M 29/00

(57) ABSTRACT

A retractor for use in surgical operations comprises a first blade, a second blade, and a third blade. In operation, the blades are initially in a closed position to assume a low profile during insertion. The blades can be independently translated and independently pivoted, thereby stretching the skin about the incision to form an aperture larger than the incision. In some embodiments, a method of performing an operation on a patient using the disclosed retractor is provided.

### CROSS-REFERENCE TO RELATED APPLICATIONS

This application claims priority benefit to U.S. Provisional Patent Application No. 62/903,318, filed Sep. 20, 2019, which is incorporated herein by reference in its entirety for all purposes.

### BACKGROUND

The present application relates to surgical methods and tools, and more particularly to a retractor and a method of operating a retractor.

### DESCRIPTION OF THE RELATED ART

Retractors are surgical devices used to spread bodily tissues in order to allow a surgeon or surgical assistant to see and access a part of the body that is to be surgically treated. In general, retractors comprise a pair of jaws or blades that grip the bodily tissue and push it apart under the force generated by an actuator, such as a pair of scissor-like arms having a distal end and a proximal end. The proximal end generally defines a pair of handles and the distal end attaches to the pair of blades so that manipulation of the handles causes the blades to move apart from one another. Once an incision is made in the body to be operated on, the blades are inserted into the incision and the actuator is manipulated to move the blades of the retractor apart, thus spreading the tissue and providing an aperture through which the surgeon can access and visualize the tissue to be surgically treated. One problem with this type of retractor is that the aperture size is generally limited by the size of the incision, meaning that a large aperture requires a relatively large incision. The drawback to this arrangement is that larger incisions result in the need for longer periods for healing of the incision. There is thus a need for a surgical retractor that is capable of creating a relatively large aperture using a relatively small incision, thereby reducing the invasiveness of the surgical procedure, post-operative healing times and patient discomfort.

### SUMMARY

In some embodiments, a retractor is provided. The retractor can include a first blade. The retractor can include a first translation mechanism that translates the first blade about a first translation axis, wherein the first translation mechanism comprises a worm gear. The retractor can include a second blade. The retractor can include a second translation mechanism that translates the second blade about a second translation axis, wherein the second translation mechanism comprises a worm gear, wherein the first blade is configured to translate independently of the second blade.

In some embodiments, the retractor can include a first pivot mechanism that tilts the first blade about a first tilt axis. In some embodiments, the retractor can include a second pivot mechanism that tilts the second blade about a second tilt axis. In some embodiments, the first blade and the second blade are symmetrical about a longitudinal axis of the retractor. In some embodiments, the retractor can include a third blade. In some embodiments, at least one of the first, second, and third blades is a curved blade. In some embodiments, the first blade, the second blade, and the third blade form a circular lumen. In some embodiments, the retractor can include a third translation mechanism that translates the third blade about a third translation axis. In some embodiments, the third translation mechanism comprises a worm gear. In some embodiments, the retractor can include a third pivot mechanism that tilts the third blade about a third tilt axis. In some embodiments, the first blade, the second blade, and the third blade are configured to tilt. In some embodiments, the first blade is configured to translate along a dovetail connection. In some embodiments, the retractor is radiolucent. In some embodiments, the first blade is configured to translate along a dovetail connection.

In some embodiments, a method of using a retractor is provided. The method can include making an incision in a tissue of a body. The method can include inserting a retractor. The method can include translating the first blade.

In some embodiments, inserting the retractor comprises inserting the retractor over a dilator. In some embodiments, the method can include translating the second blade independently. In some embodiments, the method can include pivoting the first blade. In some embodiments, translating the first blade comprises translating the first blade along a dovetail connection.

In some embodiments, a kit is provided. The kit can include a retractor. The kit can include at least one dilator, wherein the retractor is configured to slide over the dilator toward the facet joint.

### BRIEF DESCRIPTION OF THE DRAWINGS

These and other features, aspects and advantages of the described embodiments are described with reference to drawings of certain preferred embodiments, which are intended to illustrate, but not to limit. It is to be understood that the attached drawings are for the purpose of illustrating concepts of the described embodiments and may not be to scale.

FIG. 1 illustrates a perspective view of an embodiment of a retractor with blades in a closed position.

FIG. 2 illustrates another perspective view of the retractor of FIG. 1 with the blades in the closed position.

FIG. 3 illustrates a top view of the retractor of FIG. 1 with the blades in a closed position.

FIG. 4 illustrates a top view of the retractor of FIG. 1 with the top housing removed.

FIG. 5 illustrates a perspective view of the retractor of FIG. 1 with the top housing removed.

FIG. 6 illustrates an enlarged view of a translation mechanism of the retractor of FIG. 1.

FIG. 7 illustrates an exploded view of the translation mechanism of FIG. 6.

FIG. 8 illustrates an exploded view of a tilt mechanism of the retractor of FIG. 1.

FIG. 9 illustrates a perspective of a component of the tilt mechanism of FIG. 8.

FIG. 10 illustrates a perspective view of a component of the tilt mechanism of FIG. 8.

FIG. 11 illustrates an exploded view of a tilt mechanism of the retractor of FIG. 1.

FIG. 12 illustrates a perspective of a component of the tilt mechanism of FIG. 11.

FIG. 13 illustrates a perspective of a component of the tilt mechanism of FIG. 11.

FIG. 14 illustrates a perspective view of the retractor of FIG. 1 with the blades in an open position.

FIG. 15 illustrates another perspective view of the retractor of FIG. 1 with the blades in an open position.

FIG. 16 illustrates a top view of the retractor of FIG. 1 with the blades in an open position.

FIG. 17 illustrates a perspective view of the retractor of FIG. 1 with the blades in an open position.

FIG. 18 illustrates another perspective view of the retractor of FIG. 1 with the blades in an open position.

FIG. 19 illustrates a top view of the retractor of FIG. 1 with the blades in an open position.

FIG. 20 illustrates a perspective view of an embodiment of an initial dilator.

FIG. 21 illustrates a perspective view of an embodiment of a second dilator.

FIG. 22 illustrates a perspective view of an embodiment of a third dilator.

### DETAILED DESCRIPTION

As will be explained below, certain retractor embodiments described herein provide advantages over the prior art retractors. For example, the retractor of the illustrated embodiment allows a person to insert a relatively compact set of retractor blades into an incision in a closed position. In some embodiments, each blade of the compact set of retractor blades (e.g., a first blade, a second blade, a third blade) can be independently actuated. In some embodiments, each blade is translated by a self-locking translation mechanism. In some embodiments, the self-locking translation mechanism comprises a worm gear.

In some embodiments, the compact set of retractor blades form a circular lumen. The initial diameter lumen is customizable for delivery over sequential dilators. The blades can be translated to increase the size of the lumen. In some embodiments, the compact set of retractor blades provides a tube-design feature, with ability to customize the working corridor over the facet. In some embodiments, the center blade (e.g., the third blade) can comprise teeth on the distal tip of the center blade for engaging the teeth with the facet via a contralateral access approach to the facet. In some embodiments, the retractor is radiolucent comprising one or more metal materials such as aluminum. In some embodiments, one or more drive mechanisms of the retractor comprise stainless steel for more strength and wear resistance. In some embodiments, stainless steel inserts are added to the aluminum components to form the retractor. In some embodiments, the retractor can have an overall enhanced aesthetic design.

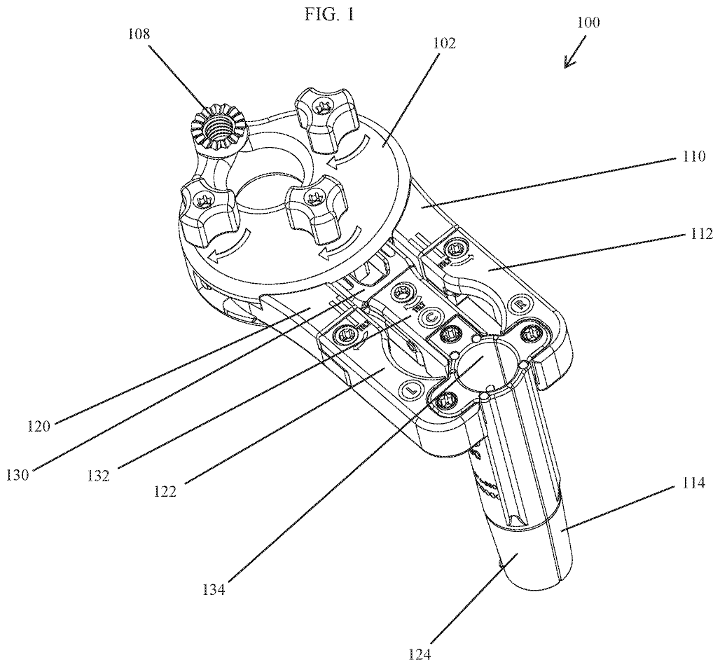

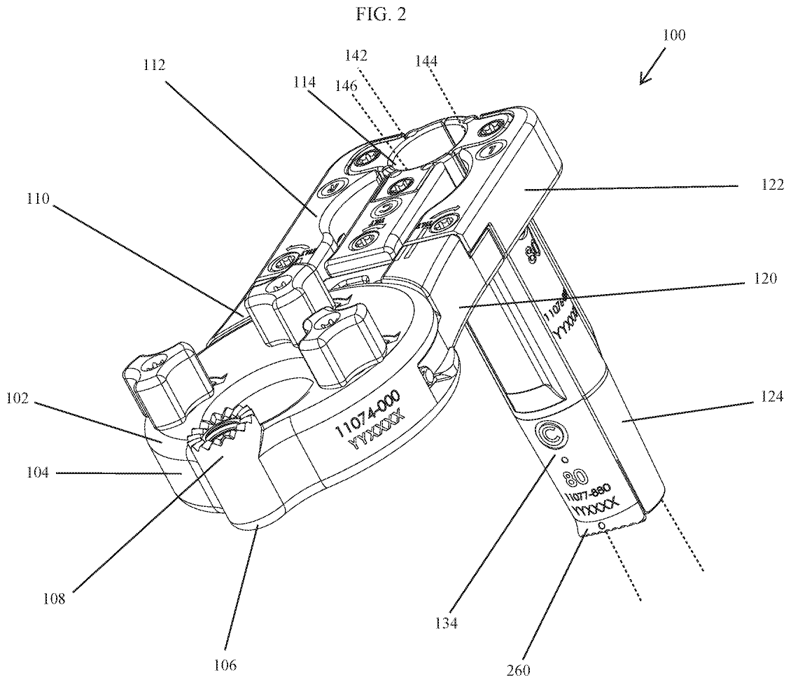

FIG. 1 is a perspective view of a retractor 100. FIG. 2 is another perspective view of the retractor 100. The retractor 100 can include a body 102. The body 102 can be any size or shape to house the translation mechanisms described herein. In the illustrated embodiment, the body is rounded or circular or oval or ellipse. The body 102 can comprises a top housing 104 and a bottom housing 106. The top housing 104 can form a covering. The bottom housing 106 can be a plate or plate like structure. The top housing 104 and the bottom housing 106 can couple through any mechanical means including one or more fasteners.

The retractor 100 can include a first arm 110. The first arm 110 can be coupled to a first blade assembly 112. The first blade assembly 112 can be coupled to a first blade 114. The retractor 100 can include a first translation mechanism 116 to translate the first blade 114. The retractor 100 can include a first tilt mechanism 118 configured to tilt the first blade 114.

The retractor 100 comprises a second arm 120. The second arm 120 can be coupled to a second blade assembly 122. The second blade assembly 122 can be coupled to a second blade 124. The retractor 100 can include a second translation mechanism 126 to translate the second blade 124. The retractor 100 can include a second tilt mechanism 128 configured to tilt the second blade 124.

The retractor 100 comprises a third arm 130. The third arm 130 can be coupled to a third blade assembly 132. The third blade assembly 132 can be coupled to a third blade 134. The retractor 100 can include a third translation mechanism 136 to translate the third blade 134. The retractor 100 can include a third tilt mechanism 138 configured to tilt the third blade 134.

The retractor 100 can include an attachment mechanism 108. The attachment mechanism 108 can be located on the body 102. The attachment mechanism 108 couples the body 102 to a fixture (not shown). The fixture can be a support arm. The fixture can be located within the operating arena. The fixture can support the body 102 during the procedure.

The attachment mechanism 108 can include features to enable coupling to the fixture. The attachment mechanism 108 can be threaded. The attachment mechanism 108 can include a serrated plate to limit rotation. The attachment mechanism 108 can include any features to ensure a stable connection between the fixture and the body 108.

The retractor 100 can include a single attachment point located at the attachment mechanism 108. The first blade 114 and the second blade 124 can move relative to the one attachment point. The first blade 114 can translate relative to the attachment point. The first blade 114 can pivot relative to the attachment point. The second blade 124 can translate relative to the attachment point. The second blade 124 can pivot relative to the attachment point. The third blade 134 can translate relative to the attachment point. The third blade 134 can pivot relative to the attachment point. Each of the blades 114, 124, 134 can move independently relative to the attachment point. Each of the blades 114, 124, 134 can move independently of the body 102.

The one attachment point located at the attachment mechanism 108 can provide more stability and accuracy during retraction. The one attachment point can maintain the position of the retractor 100 during the procedure. The surgeon does not need to switch between attachment points to allow operation of the blades 114, 124, 134. Each blade 114, 124, 134 can be manipulated when the body 102 is coupled to the fixture via the attachment mechanism 108. In some methods of use, the body 102 is not removed from the fixture during the course of the procedure.

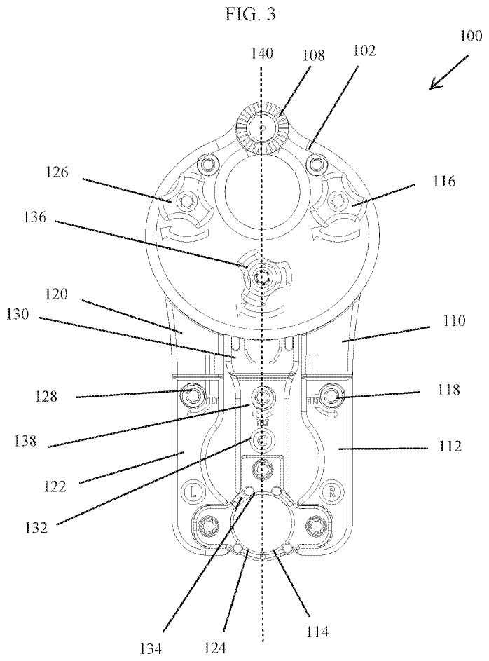

FIG. 3 is a top view of the retractor 100. The retractor 100 comprises a longitudinal axis 140. In some embodiments, the third arm 130 is aligned along the longitudinal axis 140. In some embodiments, the third blade assembly 132 is aligned along the longitudinal axis 140. In some embodiments, the third blade 134 is aligned along the longitudinal axis 140. In some embodiments, the third blade 134 is symmetric about the longitudinal axis 140.

When viewed from the top, the first arm 110, the first blade assembly 112, and the first blade 114 can be disposed on a first side of the longitudinal axis 140, for instance, the right side. When viewed from the top, the second arm 120, the second blade assembly 122, and the second blade 124 can be disposed on a second side of the longitudinal axis 140, for instance, the left side. In some embodiments, the retractor 100 can be positioned to be symmetric about the longitudinal axis 140. In some embodiments, the retractor 100 can be symmetric about the longitudinal axis 140 in the closed position. In some embodiments, the first arm 110, the first blade assembly 112, and the first blade 114 can be a mirror image of the second arm 120, the second blade assembly 122, and the second blade 124.

Each blade 114, 124, 134 can comprise a longitudinal axis. The first blade 114 can comprise a first blade axis 142. The second blade 124 can comprise a second blade axis 144. The third blade 134 can comprise a third blade axis 146. The first blade axis 142 and the second blade axis 144 can be parallel in the closed position. The first blade axis 142, the second blade axis 144, and the third blade axis 146 can be parallel in the closed position. The first blade axis 142, the second blade axis 144, and the third blade axis 146 can be vertical or substantially vertical in the closed position.

The retractor 100 is configured to transition from the closed position shown in FIG. 1 to one or more open positions. The retractor 100 can transition to an open position by translation of one or more of the blades 114, 124, 134. Each blade 114, 124, 134 can be coupled to the respective translation mechanism 116, 126, 136 configured to translate the respective blade 114, 124, 134.

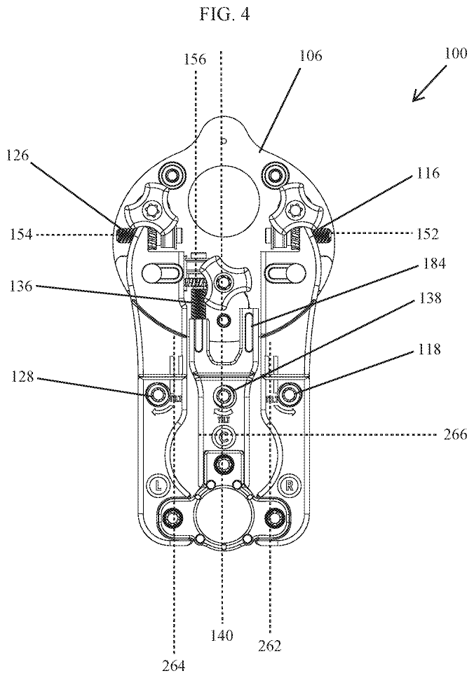

FIG. 4 is a top view of the retractor 100 with the top housing 104 removed. The top housing 104 has been removed to illustrate the first translation mechanism 116, the second translation mechanism 126, and the third translation mechanism 136.

Each translation mechanism 116, 126, 136 can comprise a translation axis. The first translation mechanism 116 can comprise a first translation axis 152. The second translation mechanism 126 can comprise a second translation axis 154. The third translation mechanism 136 can comprise a third translation axis 156. The first translation axis 152 and the second translation axis 154 can be coaxial. The first translation axis 152 and the second translation axis 154 can be parallel. The third translation axis 156 can be perpendicular to the first translation axis 152. The third translation axis 156 can be perpendicular to the second translation axis 154. The first translation axis 152, the second translation axis 154, and the third translation axis 156 can be horizontal or substantially horizontal. The first translation mechanism 116 can be vertically above or below one or more of the second translation mechanism 126 and the third translation mechanism 136. The second translation mechanism 126 can be vertically above or below one or more of the first translation mechanism 116 and the third translation mechanism 136. The third translation mechanism 136 can be vertically above or below one or more of the first translation mechanism 116 and the second translation mechanism 126. The first translation axis 152 can be vertically above or below one or more of the second translation axis 154 and the third translation axis 156. The second translation axis 154 can be vertically above or below one or more of the first translation axis 152 and the third translation axis 156. The third translation axis 156 can be vertically above or below one or more of the first translation axis 152 and the second translation axis 154.

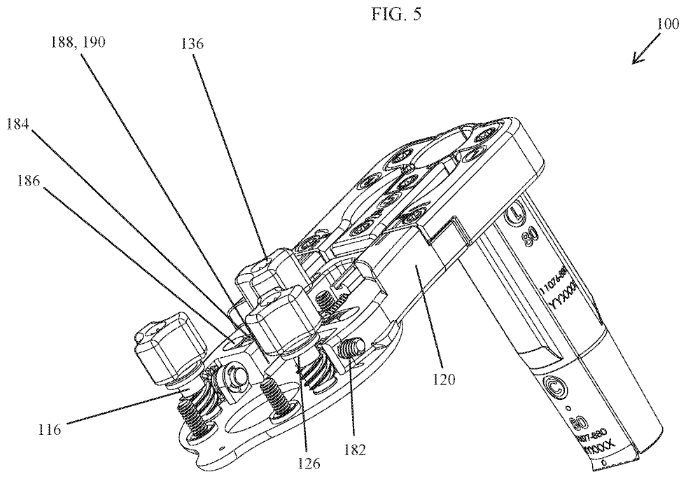

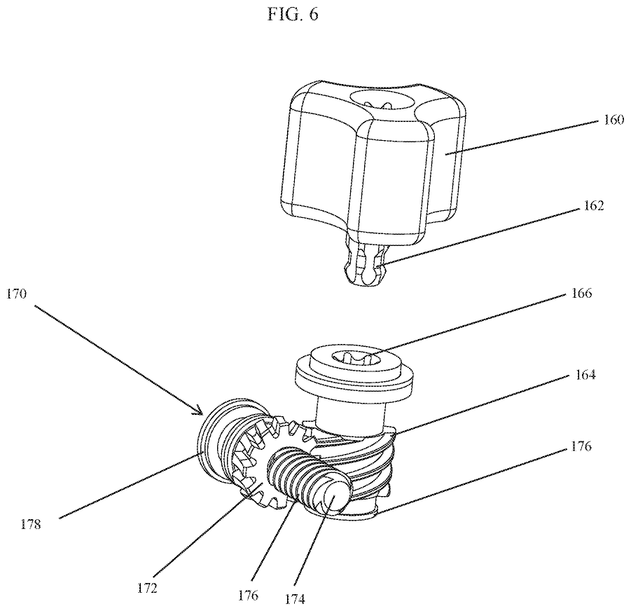

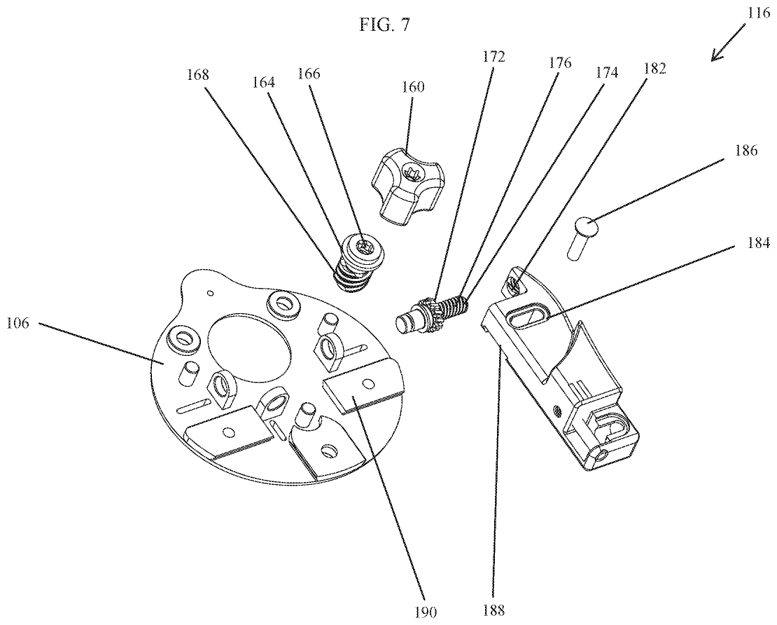

FIG. 5 is a back perspective view of the retractor 100 with the top housing 104 removed. FIG. 6 is an enlarged perspective view of the first translation mechanism 116. FIG. 7 is an exploded view of the first translation mechanism 116.

The first translation mechanism 116 can include a knob 160. The knob 160 can allow the user to easily rotate the first translation mechanism 116. The knob 160 can extend about the body 102. The knob 160 can extend into the body 102 to couple with other components of the first translation mechanism 116. The knob 160 can include a projection 162. The projection 162 can be shaped to form a mechanical interfit with a screw 164. The screw 164 can include a shaped recess 166 to receive the projection 162. The shaped recess 166 can include an interior groove to accept a canted coil. The projection 162 can be disposed within the canted coil. The canted coil can act as a retaining mechanism. The projection 162 can include a groove that engages with the canted coil. The rotation of the knob 160 can cause corresponding rotation of the screw 164. The screw 164 can include threads 168. In the illustrated embodiment, the threads 168 are right-handed threads.

The knob 160 and the screw 164 can form a worm of a worm drive. The screw 164 can be a worm screw. The first translation mechanism 116 can include a gear assembly 170. The gear assembly 170 can include a spur gear 172. The spur gear 172 can form a worm gear of a worm drive. The spur gear 172 can be a worm wheel of a worm drive.

A worm drive is a type of gear arrangement in which the worm and the worm gear rotate together. The worm and the worm gear are oriented perpendicular to each other. The drive axes are oriented 90 degrees relative to each other. In some embodiments, the worm drive can reduce the rotation speed. In some embodiments, the worm drive can transmit higher torque. In some embodiments, the worm drive can reduce the size or volume of the translation mechanism by changing the direction of the motion. The perpendicular arrangement can take up less area or volume within the body 102.

In some embodiments, the worm drive is self-locking. The direction of transmission is not self-reversible. The spur gear 172 cannot drive the screw 164. The screw 164 can only drive the spur gear 172. The reduction ratio between the spur gear 172 and the screw 164 can be large. The friction between the screw 164 and the spur gear 172 can be too high to allow for reversible transmission of the worm drive. The worm drive can be self-locking depending on one or more of the lead angle, the pressure angle, and the coefficient of friction between the spur gear 172 and the screw 164.

The gear assembly 170 can include a threaded projection 174. The rotation of the spur gear 172 can cause corresponding rotation of the threaded projection 174. The threaded projection 174 can include threads 176. In the illustrated embodiment, the threads 176 are left-handed threads. The threads 176 can be quad-lead threads to increase the speed of translation per rotation of knob 160. When viewed from the top, the threaded projection 174 extends to the right of the spur gear 172 for the first translation mechanism 116. The threaded projection 174 and the spur gear 172 can be integrally or monolithically formed. Rotation of the spur gear 172 causes rotation of the threaded projection 174.

The screw 164 can be configured to rotate without translation within the body 102. The screw 164 can include one or more retention features 176. The retention features 176 can be grooves or projections that interfit with corresponding grooves or projections in the body 102. The gear assembly 170 can be configured to rotate without translation within the body 102. The gear assembly 170 can include one or more retention features 178. The retention features 178 can be grooves or projections that interfit with corresponding grooves or projections in the body 102. The retention features 176, 178 can include bushings. The retention features 176, 178 can include clips. The retention features 176, 178 can include o-rings. The retention features 176, 178 can include retaining rings.

The first arm 110 can include a lumen 182. The lumen 182 can be threaded. The lumen 182 can couple with the threads 176 of the threaded projection 174. As the gear assembly 170 rotates, the first arm 110 translates with respect to the threaded projection 174. The first translation axis 152 can be the axis of the gear assembly 170. The first translation axis 152 can be the axis of the threaded projection 174 of the gear assembly 170. The first translation axis 152 can be the axis of the lumen 182.

During operation of the first translation mechanism 116, the knob 160 is rotated clockwise. The screw 164 rotates clockwise as the knob 160 is rotated. The screw 164 rotates the gear assembly 170 via the spur gear 172. The spur gear 172 can rotate counterclockwise. The threaded projection 174 rotates with the spur gear 172 causing the first arm 110 to translate. The first arm 110 translates along the threaded projection 174 relative to the gear assembly 170. The first arm 110 translates away from the longitudinal axis 140. The first arm 110 can include a translation slot 184. The body 102 can include a translation peg 186. The translation peg 186 can be affixed to the body 102. As the first arm 110 translates, the translation peg 186 of the body 102 is disposed within the translation slot 184 of the first arm 110. The translation peg 186 can provide support for the first arm 110 as the first arm 110 translates. The translation peg 186 can limit or prevent any movement between the first arm 110 and the body 102 other than translation of the first arm 110.

The first arm 110 can include a translation connection 188. The body 102 can include a complementary translation connection 190. The bottom housing 106 can include the complementary translation connection 190. The translation connection 188 and the complementary translation connection 190 can allow the first arm 110 to translate relative to the bottom housing 106. The peg 186 can extend from the complementary translation connection 190.

As the first arm 110 translates, the translation connection 188 of the first arm 110 slides relative to the complementary translation connection 190 of the body 102. The translation connection 188 and the complementary translation connection 190 can provide support for the first arm 110 as the first arm 110 translates. The translation connection 188 and the complementary translation connection 190 can limit or prevent any movement between the first arm 110 and the body 102 other than translation of the first arm 110. The translation connection 188 and the complementary translation connection 190 can be dovetail joint. The translation connection 188 and the complementary translation connection 190 can be any joinery technique. The translation connection 188 of the first arm 110 can be a groove or a tail of the dovetail joint. The complementary translation connection 190 of the body 102 can be a projection or a pin of the dovetail joint. The translation connection 188 and the complementary translation connection 190 can have a trapezoidal shape. The translation connection 188 and the complementary translation connection 190 are shaped to make it difficult to pull the joint apart in the vertical direction. The translation connection 188 and the complementary translation connection 190 are shaped to allow translational motion of the first arm 110.

The second translation mechanism 126 can include any of the features of the first translation mechanism 116, described herein. During operation of the second translation mechanism 126, the knob 160 is rotated clockwise. The screw 164 rotates clockwise as the knob 160 is rotated. The screw 164 rotates the gear assembly 170 via the spur gear 172. When viewed from the top, the threaded projection 174 extends to the left of the spur gear 172 for the second translation mechanism 126. The spur gear 172 rotates counterclockwise. The threaded projection 174 rotates with the spur gear 172 causing the second arm 120 to translate. The second arm 120 translates relative to the gear assembly 170. The second arm 120 translates away from the longitudinal axis 140. As the second arm 120 translates, a translation peg 186 of the body 102 slides within a translation slot 184 of the second arm 120. As the second arm 120 translates, a translation connection 188 of the second arm 120 slides relative to a complementary translation connection 190 of the body 102. The second translation axis 154 can be the axis of the gear assembly 170 of the second translation mechanism 126. The second translation axis 154 can be the axis of the threaded projection 174 of the gear assembly 170 of the second translation mechanism 126.

The third translation mechanism 136 can include any of the features of the first translation mechanism 116, described herein. During operation of the third translation mechanism 136, the knob 160 is rotated clockwise. The screw 164 rotates clockwise as the knob 160 is rotated. The screw 164 rotates the gear assembly 170 via the spur gear 172. When viewed from the top, the threaded projection 174 extends frontward of the spur gear 172 for the third translation mechanism 136. The threaded projection 174 of the third translation mechanism 136 can be parallel to the longitudinal axis 140. The spur gear 172 rotates counterclockwise. The threaded projection 174 rotates with the spur gear 172 causing the third arm 130 to translate. The third arm 130 translates relative to the gear assembly 170. The third arm 130 can translate along the longitudinal axis 140. As the third arm 130 translates, a translation peg 186 of the body 102 slides within the translation slot 184 of the third arm 130. As the third arm 130 translates, a translation connection 188 of the third arm 130 slides relative to a complementary translation connection 190 of the body 102. The complementary translation connection 190 of the body 102 corresponding to the third arm 130 can be perpendicular to the complementary translation connections 190 of the body 102 corresponding to the first arm 110 and the second arm 120. The third translation axis 156 can be the axis of the gear assembly 170 of the third translation mechanism 136. The third translation axis 156 can be the axis of the threaded projection 174 of the gear assembly 170 of the third translation mechanism 136. In some embodiments, the third translation axis 156 is parallel to the longitudinal axis 140.

Each blade 114, 124, 134 can include a worm gear for the translation mechanisms 116, 126, 136. The worm gear transmits rotational motion to a perpendicular plane or to perpendicular rotational motion. The worm gear can increase or decrease the gear ratio to fine tune the adjustment and speed of translation of the blades 114, 124, 134.

Each arm 110, 120, 130 can be connected on dovetails 188, 190. The translation connection 188 and the complementary translation connection 190 allow each arm 110, 120, 130 to smoothly translate along a fixed path. The translation connections 188, 190 can be shaped to limit deflection of the arms 110, 120, 130 and the corresponding blades 114, 124, 134 away from the body 102. The translation connections 188, 190 can be shaped to limit removal of the arms 110, 120, 130 relative to the body 102. The translation peg 186 can be shaped to limit removal of the arms 110, 120, 130 relative to the body 102.

The first blade 114 and the second blade 124 can have independent translation. In the illustrated embodiment, the translation of these side blades is not coupled. The first blade 114 can translate before the second blade 124. The first blade 114 can translate after the second blade 124. In some methods of use, the user rotates both knobs 160 associated with the first blade 114 and the second blade 124 such that the first blade 114 and the second blade 124 translate at the same time. The user can use both hands to rotate the knobs 160 simultaneously. In some embodiments, two rotational inputs are needed to translate the first blade 114 and the second blade 124.

The third blade 134 can translate towards the retractor body 102. The third blade 134 can translate away from the retractor body 102. In the illustrated embodiment, the translation of the third blade 134 is not coupled to the translation of any other blade 114, 124. The translation of the third blade 134 can be independent of the translation of the first blade 114. The translation of the third blade 134 can be independent of the translation of the second blade 124. In some methods of use, the user rotates both knobs 160 associated with the first blade 114 and the third blade 134 such that the first blade 114 and the third blade 134 translate at the same time. The user can use both hands to rotate the knobs 160 simultaneously. In some embodiments, two rotational inputs are needed to translate the first blade 114 and the third blade 134. In some methods of use, the user rotates both knobs 160 associated with the second blade 124 and the third blade 134 such that the second blade 124 and the third blade 134 translate at the same time. The user can use both hands to rotate the knobs 160 simultaneously. In some embodiments, two rotational inputs are needed to translate the second blade 124 and the third blade 134.

In the illustrated embodiment, each knob 160 is rotated in the same direction. The rotation of the respective knob 160 increases or decreases the distance of the first arm 110 from the longitudinal axis 140. The rotation of the respective knob 160 increases or decreases the distance of the second arm 120 from the longitudinal axis 140. The rotation of the respective knob 160 moves the third arm 130 along the longitudinal axis 140 relative to the body 102. In some embodiments, one or more internal components are threaded to allow for this movement when the knobs 160 are rotated. In some embodiments, translation is operated by hand. The user can rotate the knobs 160, as described herein. In some embodiments, the knobs 160 are removable relative to the screws 164. The user can remove knobs 160 to use a driver to rotate the screws 164. In some embodiments, the knobs 160 are removable via a quick release connection. The knobs 160 can be coupled to the screws 164 by any mechanical connection.

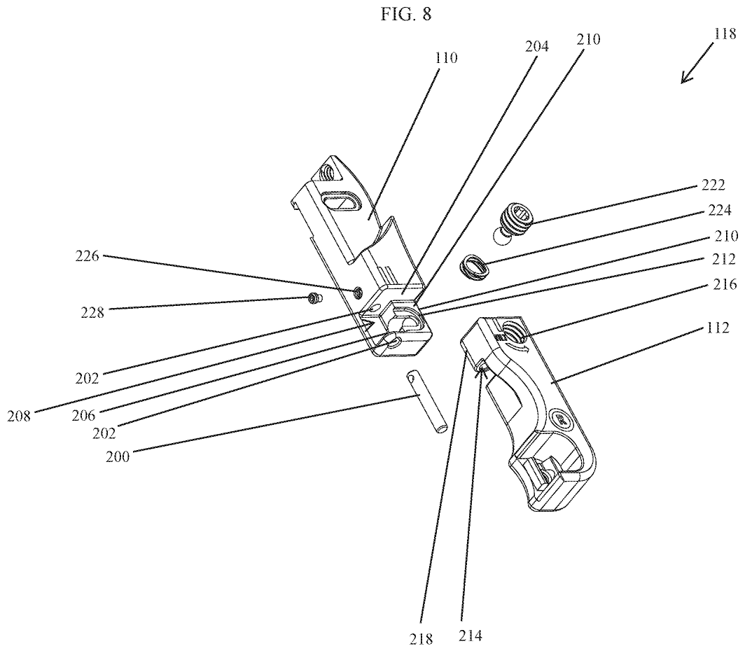

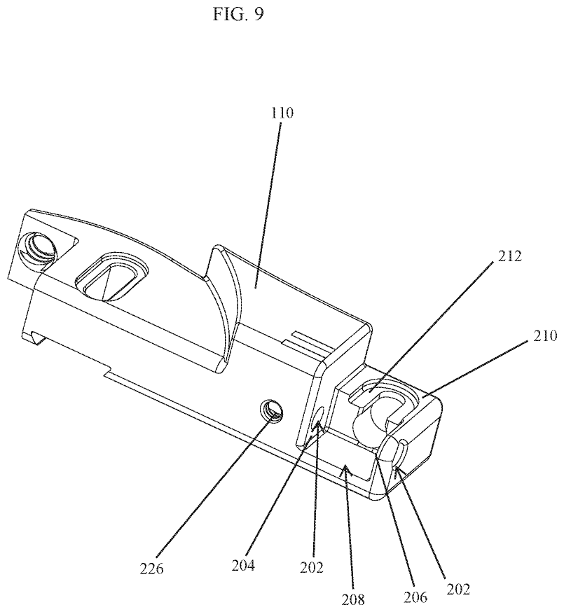

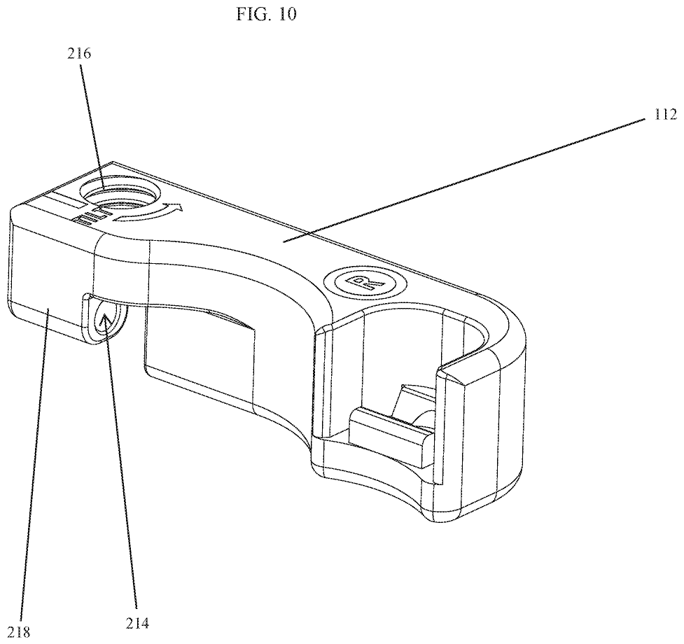

FIG. 8 is an exploded view of the first tilt mechanism 118. FIG. 9 is a perspective view of the first arm 110. FIG. 10 is a perspective view of the first blade assembly 112. The first tilt mechanism 118 includes an axle 200. The first blade assembly 112 can rotate about the axle 200 relative to the first arm 110. The first arm 110 includes a lumen 202 configured to receive a portion of the axle 200. The lumen 202 can extend longitudinally. The lumen 202 can be offset from a longitudinal axis of the first arm 110. The first arm 110 can include two opposing side walls 204, 206. The axle 200 can extend between the two opposing side walls 204, 206. The two opposing side walls 204, 206 can define a recess 208 therebetween. The axle 200 can extend in the recess 208. The two opposing side walls 204, 206 can define a hub 210 therebetween. The hub 210 can include a groove 212. The hub 210 and the recess 208 can be adjacent. The hub 210 can be an upward projection relative to the recess 208.

The first blade assembly 112 includes a lumen 214 configured to receive a portion of the axle 200. The lumen 214 can extend longitudinally. The lumen 214 can be offset from a longitudinal axis of first blade assembly 112. The first blade assembly 112 includes an actuator lumen 216. The actuator lumen 216 can be perpendicular to the lumen 214. The actuator lumen 216 can extend vertically. The actuator lumen 216 can be threaded. The first blade assembly 112 can include a hub 218. The hub 218 can include the lumen 214. The axle 200 can extend into the hub 218. The hub 218 can be a projection from the bottom surface of the first blade assembly 112. The hub 218 of the first blade assembly 112 is configured to be disposed within the recess 208 of the first arm 110. The axle 200 is configured to extend from the lumen 202 of the first arm 210 in the first side wall 204 through the lumen 214 of the first blade assembly 112, and to the lumen 202 of the first arm 210 in the second side wall 206.

The first tilt mechanism 118 can include an actuator 222. The actuator 222 can be a screw. The actuator 222 can be inserted into the actuator lumen 216. The actuator 222 is configured to be rotated. The actuator 222 can be rotated to abut the hub 210 of the first arm 110. The actuator 222 can abut the groove 212. The actuator 222 can exert a force on the hub 210. Further rotation of the actuator 222 can cause pivoting of the first blade assembly 112 relative to the first arm 110. The first blade assembly 112 rotates about the axle 200 during pivoting. As the first blade assembly 112 rotates, a spring 224 can push the first blade assembly 112 away from the first arm 110. The spring 224 can be disposed within the groove 212. The spring can be disposed between the first arm 110 and the first blade assembly 112. As the actuator 222 is rotated, the actuator 222 provides separation between the edge of the first arm 110 and the edge of the first blade assembly 112.

During operation of the first tilt mechanism 118, the actuator 222 of the first tilt mechanism 118 is rotated. The actuator 222 can push against the hub 210. The first blade assembly 112 pivots relative to the first arm 110. The first tilt axis 262 can be the axis of the axle 200 of the first tilt mechanism 118. The first tilt axis 262 can be parallel to the longitudinal axis 140.

The tilt of the first blade assembly 112 can be fixed. The first arm 110 can include a secondary lumen 226. The secondary lumen 226 can intersect the lumen 202. The secondary lumen 226 can be perpendicular to the lumen 202. The secondary lumen 226 can extend horizontally. The secondary lumen 226 can be threaded. The first tilt mechanism 118 can include a locking mechanism 228. The locking mechanism 228 can be a screw. The locking mechanism 228 can be inserted into the secondary lumen 226. The locking mechanism 228 can contact the axle 200 within the lumen 202. The locking mechanism 228 can apply friction to the axle 200 to limit or prevent tilting of the first blade assembly 112. The locking mechanism 228 can engage with axle 200 to retain axle 200 in place.

The second tilt mechanism 128 can include any of the features of the first tilt mechanism 118, described herein. During operation of the second tilt mechanism 128, the actuator 222 of the second tilt mechanism 128 is rotated clockwise. The actuator 222 can push against the hub 210. The second blade assembly 122 pivots relative to the second arm 120. As the actuator 222 is rotated, the actuator 222 provides separation between the edge of the second arm 120 and the edge of the second blade assembly 122. As the second blade assembly 122 rotates, the spring 224 can push the second blade assembly 122 away from the second arm 120. The second tilt axis 264 can be the axis of the axle 200 of the second tilt mechanism 128. The second tilt axis 264 can be parallel to the longitudinal axis 140. The first tilt mechanism 118 and the second tilt mechanism 128 can tilt in opposite directions upon clockwise rotation of the actuators 222. The first tilt mechanism 118 and the second tilt mechanism 128 can be mirror images.

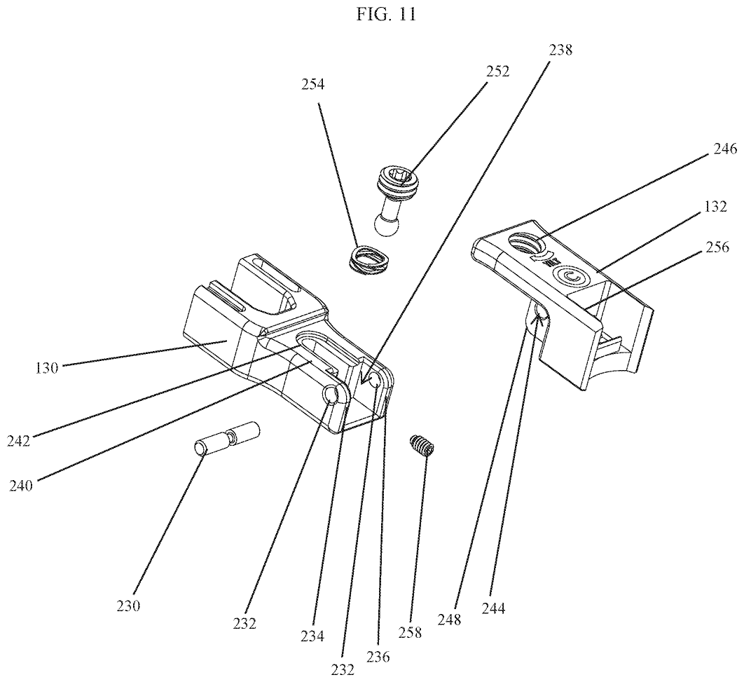

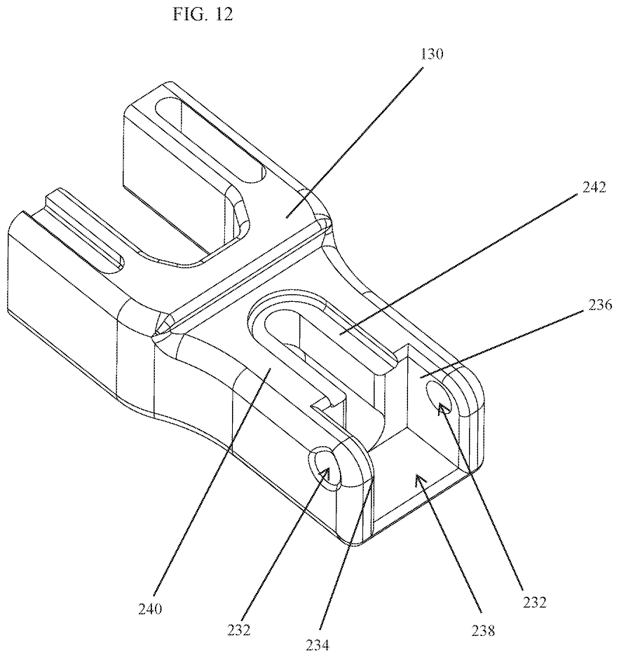

FIG. 11 is an exploded view of the third tilt mechanism 138. FIG. 12 is a perspective view of the third arm 130. FIG. 13 is a perspective view of the third blade assembly 132. The third tilt mechanism 138 can include any of the features of the first tilt mechanism 118, described herein.

The third tilt mechanism 138 includes an axle 230. The third blade assembly 132 can rotate about the axle 230 relative to the third arm 130. The third arm 130 includes a lumen 232 configured to receive a portion of the axle 230. The lumen 232 can extend perpendicular to the longitudinal axis 140. The lumen 232 can be perpendicular to a longitudinal axis of the third arm 130. The third arm 130 can include two opposing side walls 234, 236. The axle 230 can extend between the two opposing side walls 234, 236. The two opposing side walls 234, 236 can define a recess 238 therebetween. The axle 230 can extend in the recess 238. The two opposing side walls 234, 236 can define a hub 240 therebetween. The hub 240 can include a groove 242. The hub 240 and the recess 238 can be adjacent. The hub 240 can be an upward projection relative to the recess 238.

The third blade assembly 132 includes a lumen 244 configured to receive a portion of the axle 230. The lumen 244 can extend perpendicular to the longitudinal axis 140. The third blade assembly 132 includes an actuator lumen 246. The actuator lumen 246 can be perpendicular to the lumen 244. The actuator lumen 246 can extend vertically. The actuator lumen 246 can be threaded. The third blade assembly 132 can include a hub 248. The hub 248 can include the lumen 244. The axle 230 can extend in the lumen 244. The hub 248 can be a projection from the bottom surface of the third blade assembly 132. The hub 248 of the third blade assembly 132 is configured to be disposed within the recess 238 of the third arm 130. The axle 230 is configured to extend from the lumen 232 of the third arm 130 in the first side wall 234 through the lumen 244 of the third blade assembly 132, and to the lumen 232 of the third arm 130 in the second side wall 236.

The third tilt mechanism 138 can include an actuator 252. The actuator 252 can be a screw. The actuator 252 can be inserted into the actuator lumen 246. The actuator 252 is configured to be rotated. The actuator 252 can be rotated to abut the hub 240 of the third arm 130. The actuator 252 can abut the groove 242. Further rotation of the actuator 252 can cause pivoting of the third blade assembly 132 relative to the third arm 130. The third blade assembly 132 rotates about the axle 230. As the actuator 252 is rotated, the actuator 252 provides separation between the edge of the third arm 130 and the edge of the third blade assembly 132. As the third blade assembly 132 rotates, a spring 254 can push the third blade assembly 132 away from the third arm 130.

The tilt of the third blade assembly 132 can be fixed. The third blade assembly 132 can include a secondary lumen 256. The secondary lumen 256 can intersect the lumen 244. The secondary lumen 256 can be perpendicular to the lumen 244. The secondary lumen 256 can extend horizontally. The secondary lumen 256 can be threaded. The third tilt mechanism 138 can include a locking mechanism 258. The locking mechanism 258 can be a screw. The locking mechanism 258 can be inserted into the secondary lumen 256. The locking mechanism 258 can contact the axle 230 within the lumen 232. The locking mechanism 258 can apply friction to the axle 230 to limit or prevent tilting of the third blade assembly 132. The locking mechanism 258 can engage with axle 230 to retain axle 230 in place. The locking mechanism 258 can engage with a groove on axle 230 to retain axle 230 in place.

Each tilt mechanism 118, 128, 138 can comprise a tilt axis. The first tilt mechanism 118 can comprise a first tilt axis 262. The second tilt mechanism 128 can comprise a second tilt axis 264. The third tilt mechanism 138 can comprise a third tilt axis 266. The first tilt axis 262 and the second tilt axis 264 can be parallel. The third tilt axis 266 can be perpendicular to the first tilt axis 262. The third tilt axis 266 can be perpendicular to the second tilt axis 264. The first tilt axis 262, the second tilt axis 264, and the third tilt axis 266 can be horizontal or substantially horizontal.

The first blade 114 can be pivoted at any angle relative to vertical (e.g., 5°, 10°, 15°, 20°, 25°, 30°, 35°, 40°, 45°, 50°, 55°, 60°, 65°, 70°, 75°, 80°, 85°, 90°, between 10-40°, between 20-50°, between 30-60°, between 40-70°, between 50-80°, between 60-90°, or any range including and between the foregoing values). The first blade 114 can be pivoted 0° to 15° relative to vertical. The second blade 124 can be pivoted at any angle relative to vertical (e.g., 5°, 10°, 15°, 20°, 25°, 30°, 35°, 40°, 45°, 50°, 55°, 60°, 65°, 70°, 75°, 80°, 85°, 90°, between 10-40°, between 20-50°, between 30-60°, between 40-70°, between 50-80°, between 60-90°, or any range including and between the foregoing values). The second blade 124 can be pivoted 0° to 15° relative to vertical. The third blade 134 can be pivoted at any angle relative to vertical (e.g., 5°, 10°, 15°, 20°, 25°, 30°, 35°, 40°, 45°, 50°, 55°, 60°, 65°, 70°, 75°, 80°, 85°, 90°, between 10-40°, between 20-50°, between 30-60°, between 40-70°, between 50-80°, between 60-90°, or any range including and between the foregoing values). The third blade 134 can be pivoted 0° to 15° relative to vertical. The third blade 134 can be pivoted 0° to 10° relative to vertical.

The retractor 100 can allow tilt at the base of the arms 110, 120, 130. The arms 110, 120, 130 and the blade assemblies 112, 122, 132 can include the tilt mechanism 118, 128, 138 disposed therebetween. The tilt mechanism 118, 128, 138 can couple the arm 110, 120, 130 to the respective blade assembly 112, 122, 132. The first tilt mechanism 118 can allow the first blade assembly 112 to tilt relative to the first arm 110. The second tilt mechanism 128 can allow the second blade assembly 122 to tilt relative to the second arm 120. The third tilt mechanism 138 can allow the third blade assembly 132 to tilt relative to the third arm 130.

The first blade 114 and the second blade 124 can have independent tilt. In the illustrated embodiment, the pivoting of these side blades is not coupled. The first blade 114 can pivot before the second blade 124. The first blade 114 can pivot after the second blade 124. In some methods of use, the user rotates both actuators 222 associated with the first blade 114 and the second blade 124 such that the first blade 114 and the second blade 124 pivot at the same time. The user can use both hands to rotate the actuators 222 simultaneously. In some embodiments, two rotational inputs are needed to pivot the first blade 114 and the second blade 124.

The third blade 134 can pivot towards the retractor body 102. The third blade 134 can pivot away from the retractor body 102. In the illustrated embodiment, the tilt of the third blade 134 is not coupled to the tilt of any other blade 114, 124. The pivoting of the third blade 134 can be independent of the pivoting of the first blade 114. The pivoting of the third blade 134 can be independent of the pivoting of the second blade 124. In some methods of use, the user rotates the actuator 222 associated with the first blade 114 and the actuator 252 associated with the third blade 134 such that the first blade 114 and the third blade 134 tilt at the same time. The user can use both hands to rotate the actuators 222, 252 simultaneously. In some embodiments, two rotational inputs are needed to pivot the first blade 114 and the third blade 134. In some methods of use, the user rotates the actuator 222 associated with the second blade 124 and the actuator 252 associated with the third blade 134 such that the second blade 124 and the third blade 134 tilt at the same time. The user can use both hands to rotate the actuators 222, 252 simultaneously. In some embodiments, two rotational inputs are needed to pivot the second blade 124 and the third blade 134.

Each blade 114, 124, 134 can have a tilt or toe function. The first blade 114 can tilt relative to vertical. The second blade 124 can tilt relative to vertical. In some embodiments, the first blade 114 and the second blade 124 can tilt in opposite directions upon rotation of the actuators 222. In some embodiments, the first blade 114 and the second blade 124 can open the retractor 100 upon rotation of the actuators 222. In some embodiments, the first blade 114 and the second blade 124 can tilt in the same direction upon rotation of the actuators 222. The third blade 134 can tilt relative to vertical. In some embodiments, the third blade 134 can tilt in a direction perpendicular to the tilt of the first blade 114. In some embodiments, the third blade 134 can tilt in a direction perpendicular to the tilt of the second blade 124. In some embodiments, the third blade 134 can open the retractor 100 upon rotation of the actuator 252. In some embodiments, the third blade 134 can tilt toward the body upon rotation of the actuator 252.

Each actuator 222, 252 can be rotated in the same direction or a different direction, clockwise or counterclockwise. The rotation of the respective actuator 222 increases or decreases the pivot of the first blade assembly 112 relative to the first arm 110. The rotation of the respective actuator 222 increases or decreases the pivot of the second blade assembly 122 relative to the second arm 120. In some embodiments, the rotation of the respective actuators 222 can cause the first blade assembly 112 and the second blade assembly 122 to pivot in opposite directions. In some embodiments, one or more internal components are threaded to allow for this movement when the actuators 222 are rotated clockwise. The rotation of the actuator 252 increases or decreases the pivot of the third blade assembly 132 relative to the third arm 130. The third blade 130 can pivot to be closer or further away from the longitudinal axis 140.

The retractor can be symmetrical. In some embodiments, the first blade assembly 112 can be a mirror image of the second blade assembly 122. The first blade assembly 112 and the second blade assembly 122 can pivot toward each other. The configuration of the actuators 222 relative to the axles 200 allows the first blade assembly 112 to pivot in the opposite direction as the second blade assembly 122.

The first translation mechanism 116 translates the first blade 114 about the first translation axis 152. This translation is uncoupled from any other movement. The first translation mechanism 116 can translate the first blade 114 independent of the translation of any other blade. The second translation mechanism 126 translates the second blade 124 about the second translation axis 154. This translation is also uncoupled from any other movement. The second translation mechanism 126 can translate the second blade 124 independent of the translation of any other blade. The third translation mechanism 136 translates the third blade 134 about the longitudinal axis 140. This translation is also uncoupled from any other movement. The third translation mechanism 136 can translate the third blade 134 independent of the translation of any other blade. The longitudinal axis 140 and the third translation axis 156 may be substantially parallel to each other having some offset in the latitudinal direction. In some embodiments, the third translation mechanism 136 can translate the third blade 134 about the third translation axis 156. In some embodiments, the third translation axis 156 can be coaxial with the longitudinal axis 140.

In some embodiments, the first translation axis 152 is offset from the first blade 114. The first translation axis 152 does not intersect the first blade axis 142. In some embodiments, the second translation axis 154 is offset from the second blade 124. The second translation axis 154 does not intersect the second blade axis 144. In some embodiments, the first translation axis 152 and the second translation axis 154 may be substantially coaxial with one another. In some embodiments, the first translation axis 152 and the second translation axis 154 may be substantially parallel to each other having some offset in the longitudinal direction. In some embodiments, the third translation axis 156 is perpendicular to the third blade axis 146.

The first tilt mechanism 118 pivots the first blade 114 about the first tilt axis 262. This pivoting is uncoupled from any other movement. The first tilt mechanism 118 can pivot the first blade 114 independent of the pivoting of any other blade. The second tilt mechanism 128 pivots the second blade 124 about the second tilt axis 264. This pivoting is also uncoupled from any other movement. The second tilt mechanism 128 can pivot the second blade 124 independent of the pivoting of any other blade. The third tilt mechanism 138 pivots the third blade 134 about the third tilt axis 266. This pivoting is also uncoupled from any other movement. The third tilt mechanism 138 can pivot the third blade 134 independent of the pivoting of any other blade.

In some embodiments, the first tilt axis 262 is perpendicular or skewed to the first blade axis 142. In some embodiments, the second tilt axis 264 is perpendicular or skewed to the second blade axis 144. In some embodiments, the third tilt axis 266 is perpendicular or skewed to the third blade axis 146. In some embodiments, the first tilt axis 262 and the second tilt axis 264 may be substantially parallel with one another. In some embodiments, the third tilt axis 266 is perpendicular or skewed to the first tilt axis 262. In some embodiments, the third tilt axis 266 is perpendicular or skewed to the second tilt axis 264.

In the illustrated embodiment, the first arm 110 and the second arm 120 can translate relative to one another along a straight path. In the illustrated embodiment, their general direction of motion relative to one another can be along the common axis that is generally defined by a line passing through the gear assemblies 170. In other embodiments, the first blade assembly 112 and the second blade assembly 122 can translate about different axes. In some embodiments, the axes are parallel to each other. In some embodiments, the axes are slightly skewed.

The first arm 110 can be locked in position along the first translation axis 152 when the respective knob 160 is no longer rotated. The worm drive of the first translation mechanism 116 can be self-locking. One or more of the lead angle, the pressure angle, and the coefficient of friction can prevent any further translation of the first arm 110 relative to the body 102. The second arm 120 can be locked in position along the second translation axis 154 when the respective knob 160 is no longer being rotated. The third arm 130 can be locked in position along the third translation axis 156 when the respective knob 160 is no longer being rotated. In some embodiments, the first arm 110 can be self-locked at any translational position. In some embodiments, the second arm 120 can be self-locked at any translational position. In some embodiments, the third arm 130 can be self-locked at any translational position.

The first blade assembly 112 can be locked in position relative to the first arm 110 along the first tilt axis 262. The first tilt mechanism 118 can include the locking mechanism 228. The respective locking mechanism 228 can abut the axle 200 to limit or reduce further pivoting of the first blade assembly 112. The second blade assembly 122 can be locked in position relative to the second arm 120 along the second tilt axis 264. The second tilt mechanism 128 can include the locking mechanism 228. The respective locking mechanism 228 can abut the axle 200 to limit or reduce further pivoting of the second blade assembly 122. The third blade assembly 132 can be locked in position relative to the third arm 130 along the third tilt axis 266. The third tilt mechanism 138 can include the locking mechanism 258. The locking mechanism 258 can abut the axle 230 to limit or reduce further pivoting of the third blade assembly 132. The locking mechanism 258 can engage with axle 230 to retain axle 230 in place. The locking mechanism 258 can engage with a groove on axle 230 to retain axle 230 in place.

The locking mechanism 228, 258 locks with friction between two surfaces. In some embodiments, the locking mechanism 228, 258 is not self-locking. In some embodiments, the locking mechanism 228, 258 has to be manually actuated to lock. In some embodiments, the first blade assembly 112 can be locked at any rotational position. In some embodiments, the second blade assembly 122 can be locked at any rotational position. In some embodiments, the third blade assembly 132 can be locked at any rotational position

The blades 114, 124, 134 may have a variety of configurations. In some embodiments, at least one blade 114, 124, 134 is substantially rounded. In some embodiments, the inner surface of at least one blade 114, 124, 134 comprises a portion of a circle. In some embodiments, the inner surfaces of the blades 114, 124, 134 together form a complete circle. In some embodiments, the inner surface of each blade 114, 124, 134 forms an equal part of the complete circle. The inner surface of the first blade 114 can comprise 120 degrees of the arc of the circle. The inner surface of the second blade 124 can comprise 120 degrees of the arc of the circle. The inner surface of the third blade 134 can comprise 120 degrees of the arc of the circle. In some embodiments, the inner surface of each blade 114, 124, 134 forms an unequal part of the complete circle. The inner surface of the first blade 114 can comprise more than 120 degrees of the arc of the circle. The inner surface of the second blade 124 can comprise more than 120 degrees of the arc of the circle. The inner surface of the third blade 134 can comprise less than 120 degrees of the arc of the circle. In some embodiments, the inner surfaces of the blades 114, 124, 134 together form a complete ellipse. Other configurations are contemplated.

The first blade 114 can be a mirror image of the second blade 124. The third blade 134 can have a different configuration than the first blade 114. The third blade 134 can have a different configuration than the second blade 124. In some embodiments, two of the blades 114, 124, 134 are of substantially different configurations. In some embodiments, each blade 114, 124, 134 has a different configuration. In some embodiments, two of the blades 114, 124, 134 have the same length. In some embodiments, each blade 114, 124, 134 has the same or similar length. In some embodiments, each blade 114, 124, 134 has a different length.

In some embodiments, the blades 114, 124, 134 are adjacent when the blades 114, 124, 134 are in the closed position. This arrangement can allow the first blade 114 and the second blade 124 to exert force on the skin about an incision in opposing directions. This arrangement can allow the third blade 134 to exert force on the skin about an incision in a perpendicular direction.

In some embodiments, the third blade 134 comprises one or more engagement features 260. The engagement features 260 can include one or more teeth on the distal end of the third blade 134. The engagement features 260 can include one or more serrations. The engagement features 260 can include a comb-shaped structure. In some embodiments, the first blade 114 has a smooth distal end. In some embodiments, the second blade 124 has a smooth distal end.

In some embodiments, the retractor 100 can include at least one removable blade. The first blade 114 can couple to the first blade assembly 112. The first blade 114 can be removable. The second blade 124 can couple to the second blade assembly 122. The second blade 124 can be removable. The third blade 134 can couple to the third blade assembly 132. The third blade 134 can be removable.

In FIGS. 1-4, the retractor 100 is shown in the closed position, meaning that the first blade 114, the second blade 124, and the third blade 134 are aligned and relatively close to one another so as to provide a smaller cross-sectional area as compared to one or more open positions. It is understood that one or more positions may be described as closed. In some embodiments, the blades 114, 124, 134 may be aligned or substantially aligned. In some embodiments, the blades 114, 124, 134 may be touching or spaced apart. In some embodiments, the blades 114, 124, 134 form a circular or rounded lumen. In some embodiments, the blades 114, 124, 134 form an ellipse. In some embodiments, the blades 114, 124, 134 form a closed shape or a partially closed shape. In some embodiments, the blades 114, 124, 134 comprise surfaces configured to abut. The first blade axis 142, the second blade axis 144, and the third blade axis 146 can be substantially parallel or parallel in the closed position. In some embodiments, first blade axis 142, the second blade axis 144, and the third blade axis 146 can be angled or skewed to one another in the closed position. In some embodiments, one or more of the first blade 114, the second blade 124, and the third blade 134 can comprise curved triangular configurations, wherein the proximal end of one or more of the blades is wider than the distal end.

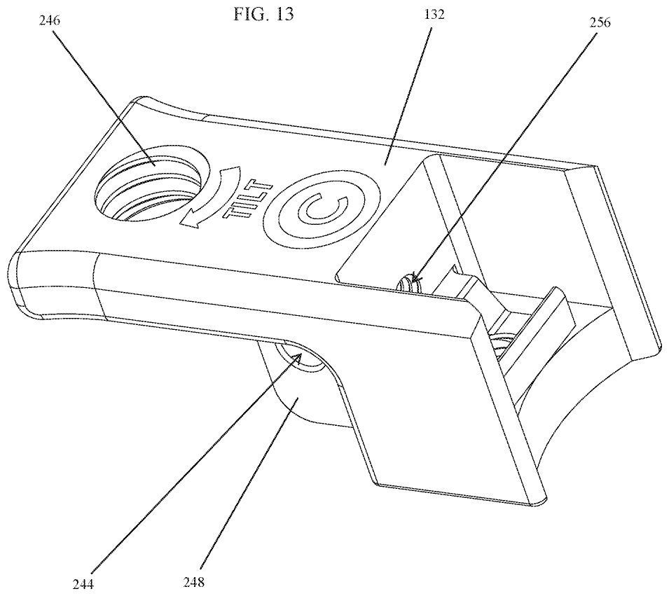

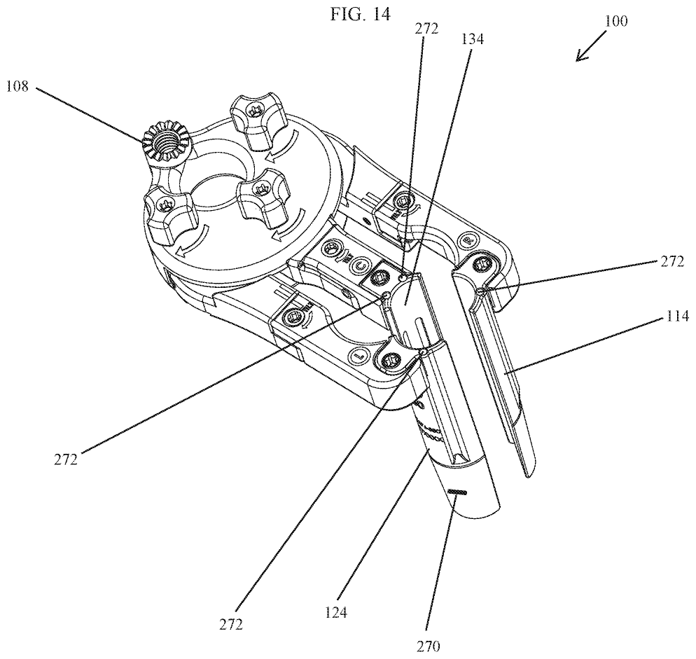

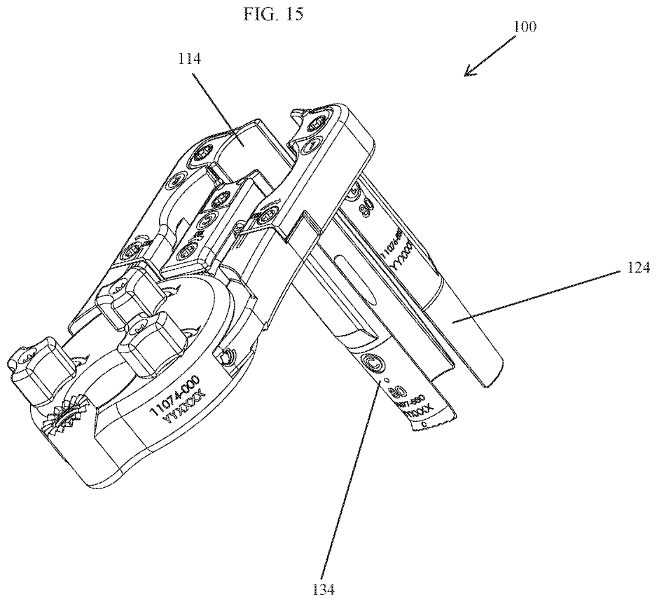

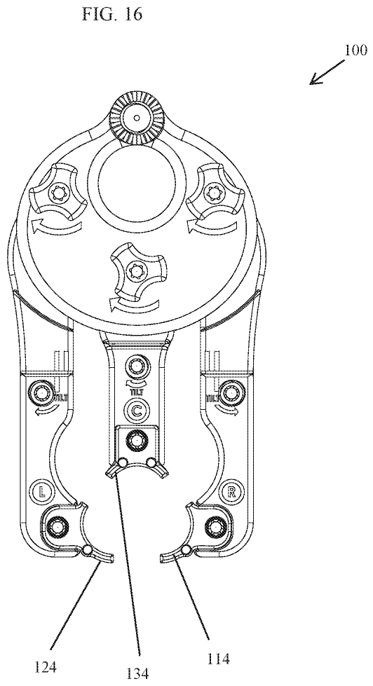

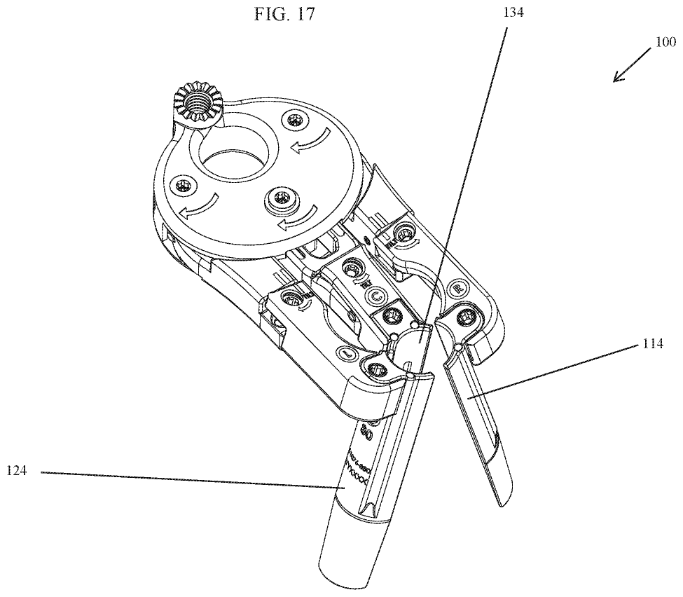

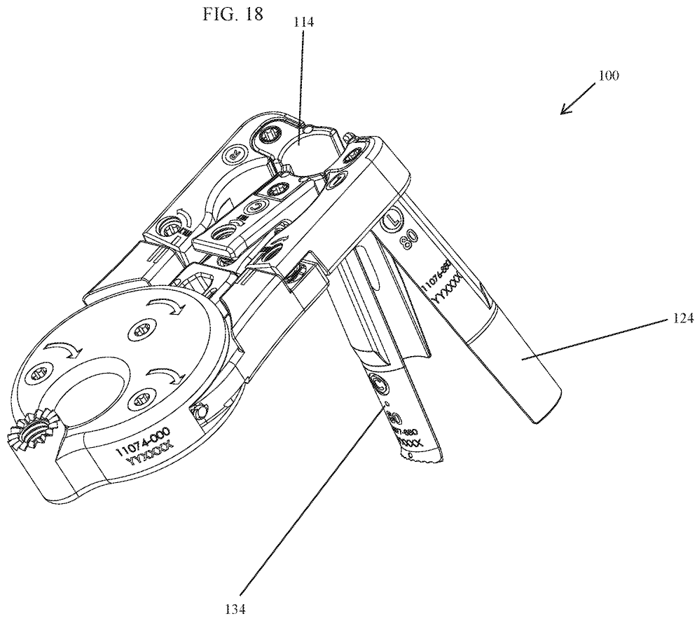

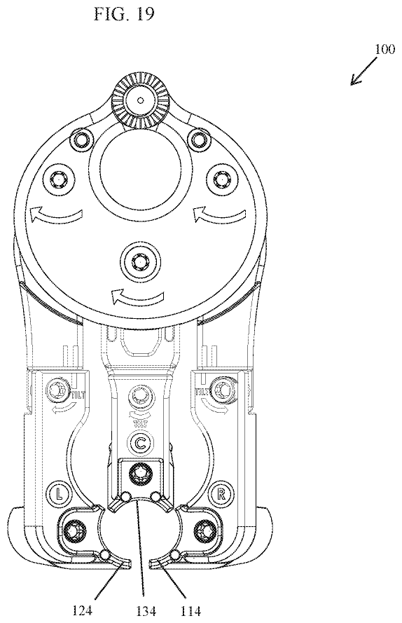

In FIGS. 14-16, the retractor 100 is shown in one of many open positions. In some embodiments, the first blade 114 is translated away from the longitudinal axis 140. In some embodiments, the second blade 124 is translated away from the longitudinal axis 140. In some embodiments, the third blade 134 is translated along the longitudinal axis 140. In FIGS. 17-19, the retractor 100 is shown in another one of many open positions. In some embodiments, the first blade 114 is pivoted. In some embodiments, the second blade 124 is pivoted. In some embodiments, the third blade 134 is pivoted. The open position can include one or more of the foregoing movements, alone or in combination. The open position can include one or more of the foregoing movements in any order of operation. The blades 114, 124, 134 can pivot before or after translating. The blades 114, 124, 134 can translate before or after pivoting.

The open position can include any additional movement that the retractor 100 is capable. One or more blades can remain stationary when one or more of the other blades move. In some embodiments, only one blade moves to transition from the closed positon to an open position. In some embodiments, only two blades move to transition from the closed positon to an open position. In some embodiments, all three blades move to transition from the closed positon to an open position. In some embodiments, two or more of the blades 114, 124, 134 may be spaced apart in an open position. In some embodiments, two or more of the blades 114, 124, 134 translate equal distance in an open position. In some embodiments, two or more of the blades 114, 124, 134 translate unequal distance in an open position. In some embodiments, two or more of the blades 114, 124, 134 equally pivot in an open position. In some embodiments, two or more of the blades 114, 124, 134 unequally pivot in an open position. In some embodiments, the blades 114, 124, 134 form a circular or rounded lumen in an open position. In some embodiments, the blades 114, 124, 134 form an oval lumen in an open position.

In the illustrated open position, the first blade 114, the second blade 124, and the third blade 134 have been translated. The first blade 114, the second blade 124, and the third blade 134 can be translated by rotating the knobs 160. In the illustrated open position, the first blade 114, the second blade 124, and the third blade 134 have been translated.

The motion of the first blade 114 can be uncoupled to the motion of the second blade 124. The motion of the first blade 114 can be uncoupled to the motion of the third blade 134. The motion of the second blade 124 can be uncoupled to the motion of the third blade 134. The first blade 114 and the second blade 124 can translate the same distance in the open position. The first blade 114 and the second blade 124 can translate different distances in the open position. The first blade 114 and the second blade 124 can pivot at the same angle in the open position. The first blade 114 and the second blade 124 can pivot at different angles in the open position. The first blade 114 and the third blade 134 can translate the same distance in the open position. The first blade 114 and the third blade 134 can translate different distances in the open position. The first blade 114 and the third blade 134 can pivot at the same angle in the open position. The first blade 114 and the third blade 134 can pivot at different angles in the open position. The second blade 124 and the third blade 134 can translate the same distance in the open position. The second blade 124 and the third blade 134 can translate different distances in the open position. The second blade 124 and the third blade 134 can pivot at the same angle in the open position. The second blade 124 and the third blade 134 can pivot at different angles in the open position.

Insertion of the blades 114, 124, 134 into an incision in the closed position (as in FIGS. 1-4) and translating or pivoting one or more of the blades 114, 124, 134 to an open position (such as in FIGS. 14-19) results in a stretching of the incision in at least one direction. This stretching can increase the size of the incision in at least one direction (e.g., length or width). This stretching can increase the circumference of the incision.

The retractor 100 can be used in any surgical procedure. In some methods of use, the retractor 100 is used in minimally invasive surgery. In some methods of use, the retractor 100 is used in transforaminal lumbar interbody fusion (TLIF). The retractor 100 can be utilized in any spinal surgery. The retractor 100 can be utilized in any approach including an anterior approach, a posterior approach, and a lateral approach. The method can include any of the steps described herein.

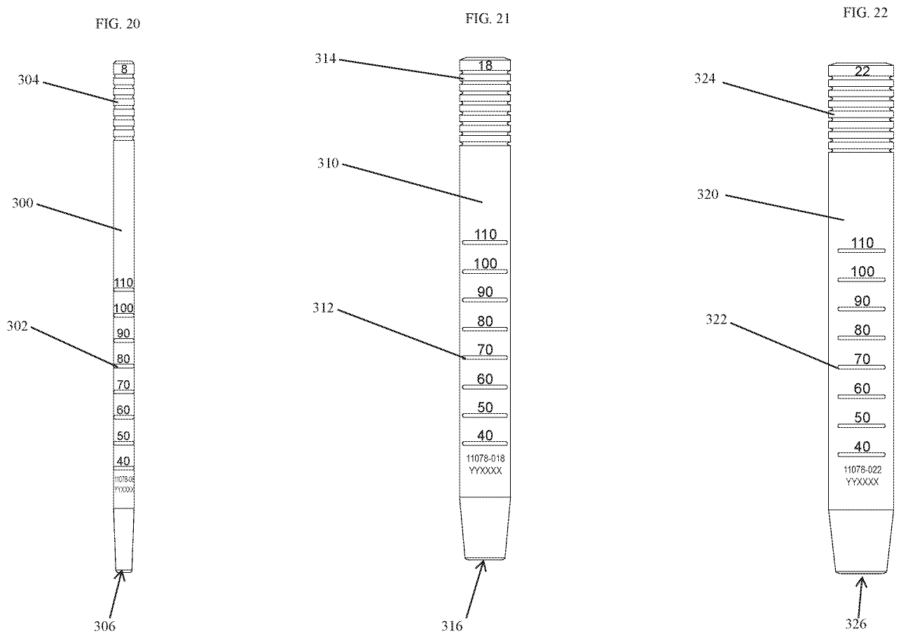

The retractor 100 can be inserted over a facet joint. In some methods of use, an initial dilator 300 is centered over or onto a facet joint. FIG. 20 illustrates the initial dilator 300. The initial dilator 300 can be a cylindrical tubular body. In some embodiments, the initial dilator 300 can be an elliptical body. The initial dilator 300 can include one or more depth markings 302. The depth markings 302 can be equally spaced. The depth markings 302 can span a range of depths. The depth markings 302 can include a marking for 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, or any range including and between the foregoing values. In the illustrated embodiment, the initial dilator 300 has depth markings 302 spanning between 40 mm and 110 mm.

The initial dilator 300 can include a handle 304. The handle 304 can include one or more knurled sections. The initial dilator 300 can include a diameter. The diameter can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, between 5 mm and 10 mm, between 7 mm and 10 mm, less than 10 mm, more than 5 mm, or any range including and between the foregoing values. In the illustrated embodiment, the initial dilator 300 has a diameter of 8 mm. The initial dilator 300 can include a lumen 306. The lumen diameter can be 1 mm, 2 mm, 3 mm, 4 mm, 5 mm, or any range including and between the foregoing values. In the illustrated embodiment, the initial dilator 300 has a lumen diameter of approximately 2 mm. The lumen 306 can be configured to accept a K-wire. The lumen 306 can be configured to assist with positing the initial dilator 300 onto a facet joint.

In some methods of use, a second dilator 310 is passed over the initial dilator 300. FIG. 21 illustrates the second dilator 310. The second dilator 310 can be a cylindrical tubular body. In some embodiments, the second dilator 310 can be an elliptical body. The second dilator 310 can include one or more depth markings 312. The depth markings 312 can be equally spaced. The depth markings 312 can span a range of depths. The depth markings 312 can including a marking for 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, or any range including and between the foregoing values. In the illustrated embodiment, the second dilator 310 has depth markings 312 spanning between 40 mm and 110 mm.

The second dilator 310 can include a handle 314. The handle 314 can include one or more knurled sections. The third dilator 320 can include a diameter. The diameter can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, between 10 mm and 20 mm, between 14 mm and 16 mm, less than 20 mm, more than 10 mm, or any range including and between the foregoing values. In the illustrated embodiment, the second dilator 310 has a diameter of 18 mm. The second dilator 310 can include a lumen 316. The lumen diameter can be 7 mm, 8 mm, 9 mm, 10 mm, 11 mm, 12 mm, or any range including and between the foregoing values. The second dilator 310 has a lumen diameter that is greater than an outer diameter of the initial dilator 300 in order for the second dilator 310 to be passed over the initial dilator 300. In the illustrated embodiment, the second dilator 310 has a lumen diameter greater than 8 mm in order to be nested over the initial dilator 300.

In some methods of use, a third dilator 320 is passed over the second dilator 310. FIG. 22 illustrates the third dilator 320. The third dilator 320 can be a cylindrical tubular body. In some embodiments, the third dilator 320 can be an elliptical body. The third dilator 320 can include one or more depth markings 322. The depth markings 322 can be equally spaced. The depth markings 322 can span a range of depths. The depth markings 312 can including a marking for 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, or any range including and between the foregoing values. In the illustrated embodiment, the second dilator 310 has depth markings 322 spanning between 40 mm and 110 mm.

The third dilator 320 can include a handle 324. The handle 324 can include one or more knurled sections. The third dilator 320 can include a diameter. The diameter can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, between 20 mm and 30 mm, between 15 mm and 25 mm, less than 30 mm, more than 20 mm, or any range including and between the foregoing values. The third dilator 320 has a lumen diameter that is greater than an outer diameter of the second dilator 310 in order for the third dilator 320 to be passed over the second dilator 310. In the illustrated embodiment, the second dilator 310 has a diameter of 22 mm. The third dilator 320 can include a lumen 326. The lumen diameter can be 14 mm, 15 mm, 16 mm, 17 mm, 18 mm, 19 mm, or any range including and between the foregoing values. In the illustrated embodiment, the third dilator 320 has a lumen diameter of greater than 18 mm in order to be nested over the second dilator 310.

The second dilator 310 and the third dilator 320 are two nesting cylindrical dilators for initial positioning. In some methods of use, the initial dilator 300 remains in place as the second dilator 310 is advanced toward the facet joint. In some methods of use, the initial dilator 300 and the second dilator 310 remain in place as the third dilator 320 is advanced toward the facet joint. In some methods of use, the initial dilator 300, the second dilator 310, and the third dilator 320 remain in place as the retractor 100 is advanced toward the facet joint. The initial dilator 300, the second dilator 310, and the third dilator 320 can be removed once the retractor 100 is centered over the facet joint.

In some methods of use, the initial dilator 300 remains in place as the second dilator 310 is advanced toward the facet joint. The initial dilator 300 can be removed. In some methods of use, the second dilator 310 remains in place as the third dilator 320 is advanced toward the facet joint. The second dilator 310 can be removed. The third dilator 320 remain in place as the retractor 100 is advanced toward the facet joint. The third dilator 320 can be removed once the retractor 100 is centered over the facet joint.

The retractor 100 can slide over the third dilator 320. The retractor 100 can include a closed position. The closed position can include an internal cross-sectional dimension created by the blades 114, 124, 134. The internal cross-sectional dimension can be a diameter if the internal cross-section is circular. The internal cross-sectional dimension can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, between 20 mm and 30 mm, between 15 mm and 25 mm, less than 30 mm, more than 20 mm, or any range including and between the foregoing values. In the illustrated embodiment, the internal cross-sectional dimension is greater than 22 mm in order to be nested over the third dilator 320. The internal cross-sectional dimension can be any dimension that allows the retractor 100 to slide over one or more dilators 300, 310, 320.

In some methods of use, only the second dilator 310 is passed over the initial dilator 300. In some methods of use, the lumen 316 of the second dilator is greater than the outer diameter of the initial dilator 300 in order for the initial dilator 300 to be nested within the second dilator 310. In some embodiments, the second dilator 310 can include a lumen diameter of 7 mm, 8 mm, 9 mm, 10 mm, 11 mm, 12 mm, or any range including and between the foregoing values. In some embodiments, the second dilator 310 has a lumen diameter greater than 8 mm in order to be nested over the initial dilator 300.

In some methods of use, only the third dilator 320 is passed over the initial dilator 300. In some methods of use, the lumen 326 of the third dilator 320 is greater than the outer diameter of the first dilator in order for the initial dilator 300 to be nested within the third dilator 320. In some embodiments, the third dilator 320 can include a lumen diameter of 7 mm, 8 mm, 9 mm, 10 mm, 11 mm, 12 mm, or any range including and between the foregoing values. In some embodiments, the third dilator 320 has a lumen diameter greater than 8 mm in order to be nested over the initial dilator 300.

In some methods of use, only the second dilator 310 is passed over the initial dilator 300 for initial positioning. In some methods of use, the initial dilator 300 can then be removed. In some methods of use, the initial dilator 300 and the second dilator 310 are removed once the retractor 100 is centered over or onto the facet joint. The retractor 100 can slide over the second dilator 310. The retractor 100 can include a closed position. The closed position can include an internal cross-sectional dimension created by the blades 114, 124, 134. The internal cross-sectional dimension can be a diameter if the internal cross-section is circular. The internal cross-sectional dimension can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, between 20 mm and 30 mm, between 15 mm and 25 mm, less than 30 mm, more than 20 mm, or any range including and between the foregoing values. In some embodiments, the internal cross-sectional dimension is greater than 18 mm in order to be nested over the second dilator 310. The internal cross-sectional dimension can be any dimension that allows the retractor 100 to slide over the second dilator 310.

In some methods of use, only the third dilator 320 is passed over the initial dilator 300 for initial positioning. In some methods of use, the initial dilator 300 can then be removed. In some methods of use, the initial dilator 300 and the third dilator 320 are removed once the retractor 100 is centered over or onto the facet joint. The retractor 100 can slide over the third dilator 320. The retractor 100 can include a closed position. The closed position can include an internal cross-sectional dimension created by the blades 114, 124, 134. The internal cross-sectional dimension can be a diameter if the internal cross-section is circular. The internal cross-sectional dimension can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, between 20 mm and 30 mm, between 15 mm and 25 mm, less than 30 mm, more than 20 mm, or any range including and between the foregoing values. In some embodiments, the internal cross-sectional dimension is greater than 22 mm in order to be nested over the third dilator 320. The internal cross-sectional dimension can be any dimension that allows the retractor 100 to slide over the third dilator 320.

The retractor 100 can include features to engage the facet. In some embodiments, the third blade 134 comprises one or more engagement features 260. The engagement features 260 can include one or more teeth on the distal end of the third blade 134. The engagement features 260 can sweep up onto the base of the facet. The engagement features 260 can be flared outward from the distal end of the third blade 134. The engagement features 260 can form a lip. The engagement features 260 can have any shape to facilitate engagement with the corresponding anatomy. In some embodiments, the engagement features 260 are located only on the third blade 134. In other embodiments, the first blade 114, the second blade 124, or both the first blade 114 and the second blade 124 can include engagement features.

The retractor 100 can include depth markings 270. FIG. 14 illustrates a line on an exterior of the second blade 124, but the depth markings 270 can include any indicia. The depth markings 270 can include any number or letter. The depth markings 270 can be on an interior or exterior portion of one or more of the blades 114, 124, 134. In some embodiments, the third blade 134 can include depth markings 270. The depth markings 270 on the retractor 100 can be equally spaced. The depth markings 270 on the retractor 100 can be unequally spaced. The depth markings 270 can span a range of depths. The depth markings 270 can include a marking for 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, or any range including and between the foregoing values. In the illustrated embodiment, the retractor has a depth marking 270 at 20 mm. The retractor 100 can include one or more depth markings 270.

The retractor 100 can include one or more lumens 272. Each lumen 272 can be sized to accept a light source. The first blade 114 can include one or more lumens 272. The second blade 124 can include one or more lumens 272. The third blade 134 can include one or more lumens 272. In the illustrated embodiment, the first blade 114 includes one lumen 272, the second blade 124 includes one lumen 272, and the third blade 134 includes two lumens 272.

The retractor 100 can include a single-point attachment. The attachment mechanism 108 can include features to enable coupling to a surgical arm, table, or other fixture. The attachment mechanism 108 can include any features to ensure a stable connection between the fixture and the body 102. In some embodiments, the retractor 100 does not have any fixation or docking to anatomical structures. In some embodiments, the retractor 100 can include one or more features to allow fixation or docking to anatomical structures. In some embodiments, the only docking point for the retractor 100 is to a surgical arm docked to an operating table.

In some embodiments, the retractor 100 can be utilized without neuromonitoring. In some embodiments, the retractor 100 can be utilized with one or more electrodes for neuromonitoring. In some embodiments, the retractor 100 translates blades 114, 124, 134 by a worm drive. In some embodiments, the retractor 100 pivots blades 114, 124, 134 by applying a force by an actuator 222, 252 to a hub.

Some embodiments contemplate kits comprising the retractor 100. In some embodiments, the kit comprises a plurality of removable and exchangeable blade assemblies 112, 122, 132. In some embodiments, the kit comprises a plurality of removable and exchangeable blades 114, 124, 134. Each kit may comprise different gear assemblies 170. In some embodiments, the kit comprises at least two pairs of identical, substantially similar, or mirror image blade assemblies. In some embodiments, the retractor 100 may be provided to a surgeon or surgical personnel in the form of a kit comprising additional surgical articles and optionally instructions for the use and handling of the retractor.

While certain embodiments have been shown and described herein, it will be clear to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A retractor comprising:
a first blade;
a first translation mechanism that translates the first blade about a first translation axis, wherein the first translation mechanism comprises a worm gear;
a second blade;
a second translation mechanism that translates the second blade about a second translation axis, wherein the second translation mechanism comprises a worm gear, wherein the first blade is configured to translate independently of the second blade.

2. The retractor of claim 1, further comprising a first pivot mechanism that tilts the first blade about a first tilt axis.

3. The retractor of claim 2, further comprising a second pivot mechanism that tilts the second blade about a second tilt axis.

4. The retractor of claim 1, wherein the first blade and the second blade are symmetrical about a longitudinal axis of the retractor.

5. The retractor of claim 1, further comprising a third blade.

6. The retractor of claim 5, wherein at least one of the first, second, and third blades is a curved blade.

7. The retractor of claim 5, wherein the first blade, the second blade, and the third blade form a circular lumen.

8. The retractor of claim 5, further comprising a third translation mechanism that translates the third blade about a third translation axis.

9. The retractor of claim 8, wherein the third translation mechanism comprises a worm gear.

10. The retractor of claim 5, further comprising a third pivot mechanism that tilts the third blade about a third tilt axis.

11. The retractor of claim 5, wherein the first blade, the second blade, and the third blade are configured to tilt.

12. The retractor of claim 5, wherein the first blade is configured to translate along a dovetail connection.

13. The retractor of claim 1, wherein the retractor is radiolucent.

14. The retractor of claim 1, wherein the first blade is configured to translate along a dovetail connection.

15. A method of using a retractor, comprising:
making an incision in a tissue of a body;
inserting the retractor of claim 1;
translating the first blade.

16. The method of claim 15, wherein inserting the retractor comprises inserting the retractor over a dilator.

17. The method of claim 15, further comprising translating the second blade independently.

18. The method of claim 15, further comprising pivoting the first blade.

19. The method of claim 15, wherein translating the first blade comprises translating the first blade along a dovetail connection.

20. A kit comprising:
the retractor of claim 1;
at least one dilator, wherein the retractor is configured to slide over the dilator toward the facet joint.
